SEQUENCE LISTING (19 sequences)

SEQ ID NO: 1 - DNA, 1933 bp, Gossypium arboreum
ccacttcgcagcaatattattgcagttcctggttggctacctctgagttttcaacttaaaatttcttggttttcctcaagaaggaagaagatgttgcaaatagctttcagctcgtattcatggctgttgactgctagcaaccagaaagatggaatgttgttcccagtagctttgtcatttttggtagccatattgggaatttcactgtggcacgtatggaccataaggaagccaaagaaagacatcgccccattaccgccgggtccccgtgggttgccaatagtgggatatcttccatatcttggaactgataatcttcacttggtgtttacagatttggctgcagcttacggtcccatctacaagctttggctaggaaacaaattatgcgtagtcattagctcggcaccactggcgaaagaagtggttcgtgacaacgacatcacattttctgaaagggatcctcccgtttgtgcaaagattattacctttggcctcaatgatattgtatttgattcttacagtagtccagattggagaatgaagagaaaagtgctggtacgtgaaatgcttagccatagtagcattaaagcttgttatggtctaaggagggaacaagtgcttaaaggcgtacaaaatgttgctcaaagtgctggcaagccaattgattttggtgaaacggcatttttaacatcaatcaatgcgatgatgagcatgctgtggggtggcaaacagggaggagagcggaaaggggccgacgtttggggccaatttcgagatctcataaccgaactaatggtgatacttggaaaaccaaacgtttctgatattttcccggtgcttgcaaggtttgacatacagggattggagaaggaaatgactaaaatcgttaattctttcgataagcttttcaactccatgattgaagaaagagagaactttagcaacaaattgagcaaagaagatggaaacactgaaacaaaagacttcttgcagcttctgttggacctcaagcagaagaacgatagcggaatatcgataacaatgaatcaagtcaaggccttgctcatggacattgtggtcggtggaactgatacaacatcaaccatgatggaatggacaatggctgaactaattgcaaatcctgaagcaatgaaaaaggtgaagcaagaaatagacgatgttgtcggttcggatggcgccgtcgatgagactcacttgcctaagttgcgctatctagatgctgcagtaaaggagaccttccgattgcacccaccgatgccactccttgtaccccgttgcccgggcgactcaagcaacgttggtggctatagcgtaccaaagggcaccagggtcttcttaaacatttggtgtattcagagggatccacagctttgggaaaatcctttagaattcaagcctgagaggttcttgactgatcatgagaagctcgattatttaggaaacgattcccggtacatgccgtttggttctggaaggagaatgtgtgccggagtatctctcggtgaaaagatgttgtattcctccttggcagcaatgatccatgcttatgattggaacttggccgacggtgaagaaaatgacttgattggcttatttggaattattatgaagaaaaagaagcctttaattcttgttcctacaccaagaccatcaaatctccagcactatatgaagtaactttactattgtatttcttttataccactttattgcctctttgtcatgtttaggcaacaattctaagtaataagtttggctatatggtgaacaataatgtgtttattatacatcataagcaatgagctcttcccgaccctagggcaatacaatgatactgtgtattaagtgaaatcaacaaatcttttattctaaaaaaaaaaaaaaaaaaaaaaaaaaaaaaaaaaaaaaa

SEQ ID NO: 2 - protein, 535 aa, Gossypium arboreum
MLQIAFSSYSWLLTASNQKDGMLFPVALSFLVAILGISLWHVWTIRKPKKDIAPLPPGPRGLPIVGYLPYLGTDNLHLVFTDLAAAYGPIYKLWLGNKLCVVISSAPLAKEVVRDNDITFSERDPPVCAKIITFGLNDIVFDSYSSPDWRMKKKVLVREMLSHSSIKACYGLRREQVLKGVQNVAQSAGKPIDFGETAFLTSINAMMSMLWGGKQGGERKGADVWGQFRDLITELMVILGKPNVSDIFPVLARFDIQGLEKEMTKIVNSFDKLFNSMIEERENFSNKLSKEDGNTETKDFLQLLLDLKQKNDSGISIMNQVKALLMDIVVGGTDTTSTMMEWTMAELIANPEAMKKVKQEIDDVVGSDGAVDETHLPKLRYLDAAVKETFRLHPPMPLLVPRCPGDSSNVGGYSVPKGTRVFLNIWCIQRDPQLWENPLEFKPERFLTDHEKLDYLGNDSRYMPFGSGRRMCAGVSLGEKMLYSSLAAMIHAYDWNLADGEENDLIGLFGIIMKKKKPLILVPTPRPSNLQHYMK

SEQ ID NO: 3 - protein, 516 aa, Arabidopsis thaliana
MSPISNLFPDNTINLTPYAIVILTTVFSILWYIFKRSPQPSLPPGPRGLPIVGNLPFLDPDLHTYFANLAQSHGPIFKLNLGSKLTIVVNSPSLAREILKDQDINFSNRDVPLTGRAATYGGIDIVWTPYGAEWRQLKKICVLKLLSRKTLDSFYELRRKEVRERTRYLYEQGRKQSPVKVGDQLFLTMMNLTMNMLWGGSVKAEEMESVGTEFKGVISEITRLLSEPHVSDFFPWLARFDLQGLVKRMGVCARELDAVLDRAIEQMKPLRGRDDDEVKDFLQYLMKLKDQEGDSEVPITINHVKALITDMVVGGTDTSTNTIEFAMAELMSNPELIKRAQEELDEVVGKDNIVEESHITRLPYILAIMKETLRLHPTLPLLVPHRPAENTVVGGYTIPKDTKIFVNVWSIQRDPNVWENPTEFRPERFIDNNSCDFTGANYSYFPFGSGRRICAGVALAERMVLYTLATLLHSFDWKIPEGHVLDLKEKFGIVLKLKIPLVALPIPRFSDSNLYL

SEQ ID NO: 4 - protein, 520 aa, Arabidopsis thaliana
MSMLSNLFPDNAISLTPYAYAVLILTATFSILWYIFKRSPQPPLPPGPRGLPIVGNLPFLDPDLHTYETKLAQSHGPIFKINLGSKLTVVVNSPSLASEILKDQDINFSNHDVPLTARAVTYGGLDLVWLPYGAEWRMLRKVCAAKLFSRKTLDSFYELRRKEIRERTRCLYQKGLEKSPVNVGEQLFLTMMNLMMNMLWGGSVKAEDMESVGTEFKGVISEITRLLGVPNVSDFFPMLARFDLQGLVKKMHLYARDLDAILDRAIEQMQRLRSRDGDDGECKDFLQHLMKLRDQEADSDVPITMNHVKAVLMDMVVGGTESSTNTIEFVMAELISNPELMRRAQQELDEVVGKDNIVEESHITSLPYILAVLKSTLRLYPTIPLLVPHRPSETALVGGYTIPKNTKIFINVWSIQRDPNVWEYPTEFRPERFLDKKSCDFTGTDYSYLPFGSGRRICAGIALAERMILYTLATLLHSFDWTIPDGHVLDLEEKFGIVLKLTKPLVALPIPRLSNSNFYF

SEQ ID NO: 5 - DNA, 25 bp, Artificial Sequence
  degenerate primer
gcggatccgarttymgnccngarmg

SEQ ID NO: 6 - protein, 8 aa, Artificial Sequence
  synthesized conserved peptide sequence
EEFXPERF

SEQ ID NO: 7 - DNA, 19 bp, Artificial Sequence
  primer
tgactgatcatgagaagct

SEQ ID NO: 8 - DNA, 19 bp, Artificial Sequence
  primer
gtgctggagatttgatggt

SEQ ID NO: 9 - DNA, 20 bp, Artificial Sequence
  primer
cacatccmttcgattccgac

SEQ ID NO: 10 - DNA, 20 bp, Artificial Sequence
  primer
aggcttaaatggtgggtggt

SEQ ID NO: 11 - DNA, 20 bp, Artificial Sequence
  primer
gaagcctcatcgataccgtc

SEQ ID NO: 12 - DNA, 19 bp, Artificial Sequence
  primer
ctaccactaccatcatgtc

SEQ ID NO: 13 - DNA, 27 bp, Artificial Sequence
  primer
gggtaccatgttgcaaatagctttcag

SEQ ID NO: 14 - DNA, 28 bp, Artificial Sequence
  primer
gggagctcttacttcatatagtgctgga

SEQ ID NO: 15 - protein, 10 aa, Gossypium arboreum
FGSGRRMCAG

SEQ ID NO: 16 - protein, 8 aa, Gossypium arboreum
  VARIANT 1: Xaa = Proline or Inosine
XPGPXXXP

SEQ ID NO: 17 - protein, 8 aa, Gossypium arboreum
PPGPRGLP

SEQ ID NO: 18 - protein, 6 aa, Gossypium arboreum
  VARIANT 1: Xaa = Alanine or Glycine
XGXXTX

SEQ ID NO: 19 - protein, 6 aa, Gossypium arboreum
GGTDTT

(19) United States
(12) Patent Application Publication
(10) Pub. No.: US 2002/0187538 A1
(43) Pub. Date: Dec. 12, 2002

(54) cDNA clone of (+)- delta-cadinene-8-hydroxylase gene from cotton plants

(76) Inventors: Margaret K. Essenberg, Stillwater, OK (US); Xiao-Ya Chen, Shanghai (CN); Ping Luo, Lexington, KY (US); Yan-Hong Wang, Stillwater, OK (US)

Correspondence Address: BURNS DOANE SWECKER & MATHIS L L P, POST OFFICE BOX 1404, ALEXANDRIA, VA, 22313-1404, US

(21) Appl. No.: 10/067,534

(22) Filed: Feb. 7, 2002

Related U.S. Application Data
(60) Provisional application No. 60/267,160, filed on Feb. 7, 2001.

(51) Int. Cl. C12N 9/04; C07H 21/04

(52) U.S. Cl. 435/190; 536/23.2; 435/320.1

(57) ABSTRACT

The present invention relates to a gene, derived from cotton plants, which encodes (+)-γ-cadinene 8-hydroxylase. The present invention further relates to a vector which expresses such a gene, and to polypeptides expressed by said gene.

### FIELD OF THE INVENTION

This invention relates to the isolation and cloning of a gene derived from cotton plants and the use of the enzyme therefrom in the biosynthesis pathway of gossypol. This invention also relates to a vector comprising said gene.

### BACKGROUND OF THE INVENTION

The cultivated species of cotton (Gossypium hirsutum and G. barbadense) synthesizes a group of sesquiterpenoids, gossypol and related compounds that defend the plants against microbial pathogens and animal herbivores. Cottonseed contains these compounds in dark-colored glands within the embryo. Since they are toxic to livestock and to humans their presence limits the use of cottonseed for feed and fuel. The amino acid composition of cottonseed meal approaches that of milk protein in quality, and the meal has a pleasantly mild flavor. Cottonseed oil is extracted by a process that separates it from the sesquiterpenes, but it is not economically feasible to remove the sesquiterpenes from cottonseed meal. Crushed cottonseed is used in cattle feed, but only at low rates because of its toxic sesquiterpene content. Thus, if cotton varieties lacking toxic sesquiterpenes in their seeds could be developed, cottonseed would have greater value for feed and could also be used as a nutritious food for humans.

Since the gossypol-related sesquiterpene are important in disease and pest resistance of cotton plants there is a need to develop cotton plants that are genetically blocked in biosynthesis of the gossypol-like compounds in their seeds, but produce these compounds normally in all other parts of the plant.

The first committed step in gossypol synthesis is the cyclization of trans, trans-farnesyl diphosphate to (+)-ä-cadinene. Clones for this product have been developed by Chen et al. and have been used in an effort to genetically block this step by anti-sense suppression and by co-suppression, using a seed specific promoter to regulate the transgenes. This approach has provided a suppression of only about 40%. A problem for suppression of this step is that cotton possesses two types of (+)-ä-cadinene genes, A and C, which are only 80% identical at the amino acid level. Moreover, diploid G. arboreum has six copies of the C gene while the cultivated allotetraploid cotton species G. hirsutum has twelve copies. Thus no single transgene is perfect for suppressing the expression of all copies of genes for (+)-ä-cadinene synthase.

It is thus an object of the present invention to provide a gene construct which is effective for the suppression of biosynthesis of gossypol and related sesquiterpenes.

Another object of the present invention is to provide cotton cultivates which avoid the presence of sesquiterpenoids in the seeds thereof.

A still further object of the present invention is to provide a cottonseed product which is suitable for use as a feed for both livestock and humans.

### SUMMARY OF THE INVENTION

The present invention is based upon the inventors discovery and cloning of a gene for (+)-ä-cadinene 8-hydroxylase. This product is considered to be the second committed step of gossypol biosynthesis and is present in G. arboreum as a single copy. Thus, a significant advantage for suppression of biosynthesis of gossypol and related sesquiterpenes is provided since a transgene in anti-sense or sense orientation could be used which would be a perfect match to the native gene whose expression is sought to be suppressed.

The cDNA encodes a protein of 536 amino acid residues with a calculated molecular mass of 60.1 kDa. The sequence of the protein, as well as its sensitivity to inhibition by carbon monoxide, clotrimazole and miconazole indicates that it is a cytochrome P450. The cDNA has been classified as CYP706B1.

The cDNA was isolated from G. arboreum and was cloned in yeast, Saccharomyces cervisiae strain WR in the expression vector pYeDP60 (Urban et al., 1994).

Thus, the present invention relates to a DNA fragment comprising the sequence of SEQ ID NO: 1.

The present invention further relates to a DNA fragment which is at least 60, 65, 70, 75, 80, 85, 90, or 95% homologous to the sequence of SEQ ID NO: 1.

The present invention also relates to a polypeptide sequence which comprises the amino acid sequence identified as CYP706B1 in FIG. 2.

The present invention further relates to a polypeptide sequence which is at least 60, 65, 70, 75, 80, 85, 90, or 95% homologous to the sequence identified as CYP706B1 in FIG. 2, and which has the same biological activities of said sequence.

The present invention also relates to a vector for transforming a plant, such as cotton, specifically a cotton seed, which comprises a DNA fragment which comprises the sequence of SEQ ID NO:1, or a DNA fragment which is at least 60, 65, 70, 75, 80, 85, 90, or 95% homologous to the sequence of SEQ ID NO: 1.

A better understanding of the present invention, its several aspects, and its objects and advantages will become apparent to those skilled in the art from the following detailed description, taken in conjunction with the attached drawings, wherein there is shown and described the preferred embodiment of the invention, simply by way of illustration of the best mode contemplated for carrying out the invention. All references cited herein are incorporated by reference in their entirety.

### BRIEF DESCRIPTION OF THE DRAWINGS

FIG. 1 is an RT-PCR analysis (30 cycles of amplification) of expression of LP132 and CAD1-C in developing seeds (27 DPA), petals and pericarp (−3 DPA) of glanded and glandless cotton cultivars. His3, amplification with histone3-specific primers as an internal control; +, positive control with corresponding plasmid DNA as the template; −, negative control, no template DNA; g, glanded cultivar of G. hirsutum cv. Zhong-12; gl, glandless cultivar of G. hirsutum cv. Hai-1.

FIG. 2 is an alignment of the deduced amino acid sequence of CYP706B1 of G. arboreum with those of CYP706A4 and CYP706A5 of A. thaliana. Consensus sequences discussed are underlined. The region used to synthesize the degenerate primer is also underlined. Its sequence in CYP706B1 turned out to be slightly different from the primer.

FIG. 3 is a Southern blot of CYP706B1 in genome of Gossypium arboreum L. DNA of 20 mg was digested with Xba I, EcoR V and EcoR I, separated on an 0.8% agarose gel, and hybridized with a ³²P-labeled CYP706B1 probe.

FIG. 4 is a reversed phase HPLC of reaction mixtures. [³H] (+)-d-cadinene was incubated with yeast microsomes containing CYP706B1 (A) or another cotton clone LP64 (B). [³H] (+)-d-cadinene eluted at 60 min; the product eluted at 33.5 min.

FIG. 5 provides mass spectrum of the product obtained by incubation of (+)-d-cadinene with yeast microsomes containing CYP706B1 (A), proposed fragmentation scheme of the molecular ion (B), and the reaction catalyzed by the cotton P450 monooxygenase, CYP706B1.

FIG. 6 provides an RT-PCR analysis (30 cycles of amplification) of CYP706B1 expression in cotton seedlings (A). Lanes 1-3: roots, hypocotyls and cotyledons, respectively, of the glanded cultivar G. arboreum; lanes 4-6: roots, hypocotyls and cotyledons, respectively, of the glandless cultivar G. hirsutum cv. Hai-1; Northern blot of CYP706B1 transcripts in roots of cotton seedlings (B). Lane 1, G. arboreum; lane 2, G. hirsutum cv. Hai-1; sesquiterpene aldehydes in different tissues of cotton seedlings (C). Lane 1, roots of G. hirsutum cv. Hai-1; lanes 2-4, roots, hypocotyls and cotyledons, respectively, of G. arboreum.

FIG. 7 provides an RT-PCR analysis (25 cycles of amplification) of CYP706B1 and CAD1-C in developing seeds of G. arboreum (A); sesquiterpene aldehyde accumulation in the seeds (B).

FIG. 8 shows induced expression of CYP706B1 and accumulation of sesquiterpene aldehydes in G. arboreum suspension cultured cells treated with V. dahliae elicitors. Northern blot of CYP706B1 transcripts (A); sesquiterpene aldehydes (B).

### DETAILED DESCRIPTION OF THE INVENTION

Before explaining the present invention in detail, it is important to understand that the invention is not limited in its application to the details of the construction illustrated and the steps described herein. The invention is capable of other embodiments and of being practiced or carried out in a variety of ways. It is to be understood that the phraseology and terminology employed herein is for the purpose of description and not of limitation.

In the context of the coding sequences and genes of this invention, “homologous” refers to genes whose expression results in expression products which have a combination of amino acid sequence similarity or identity (or base sequence similarity for transcript products) and functional equivalence, and are therefore homologous genes. In general such genes also have a high level of DNA sequence similarity (i.e., greater than 80% when such sequences are identified among members of the same genus, but lower when these similarities are noted across fungal genera), but are not identical. Preferred genetic homologs include those genes which are about at least 85%, 90% or 95% similar at the nucleic acid or the amino acid level. The combination of functional equivalence and sequence similarity means that if one gene is useful, e.g., as a target for an antifungal agent, or for screening for such agents, then the homologous gene is likewise useful. In addition, identification of one such gene serves to identify a homologous gene through the same relationships as indicated above.

Due to the DNA sequence similarity, homologous genes are often identified by hybridizing with probes from the initially identified gene under hybridizing conditions which allow stable binding under appropriately stringent conditions (e.g., conditions which allow stable binding with at least approximately 85% or more sequence identity). Hybridization methods are known in the art and include, but are not limited to: (a) washing with 0.1X SSPE (0.62 M NaCl, 0.06 M NaH₂PO₄.H₂O, 0.075 M EDTA, pH 7.4) and 0.1% sodium dodecyl sulfate (SDS) at 50° C.; (b) washing with 50% formamide, 5X SSC (0.75 M NaCl, 0.075 M sodium citrate), 50 mM sodium phosphate (pH 6-8), 0.1% sodium pyrophosphate, 5X Denhardt's solution, sonicated salmon sperm DNA (50 μg/ml), 0.1% SDS and 10% dextran sulfate at 42° C., followed by washing at 42° C. in 0.2X SSC and 0.1% SDS; and (c) washing with of 0.5 M NaPO₄, 7% SDS at 65° C. followed by washing at 60° C. in 0.5X SSC and 0.1% SDS. High stringency hybridization conditions are those performed at about 20° C. below the melting temperature (Tₘ) of the probe. Preferred stringency is performed at about 5-10° C. below the melting temperature (Tₘ) of the probe. Additional hybridization conditions can be prepared as described in Chapter 11 of Molecular Cloning: A Laboratory Manual, 2nd Ed., ed. By Sambrook, Fritsch and Maniatis (Cold Spring Harbor Laboratory Press: 1989), or as would be known to the artisan of ordinary skill. The equivalent function of the product is then verified using appropriate biological and/or biochemical assays.

One of skill in the art of plant molecule biology will understand that the term “transgenic plant” means a new plant created by introducing an isolated DNA into the genome of the starting plant. The term “gene” means a nucleic acid molecule, which is usually a DNA molecule, but can also be an RNA molecule. The nucleic acid molecule may be a DNA fragment which encodes a protein which is expressed by the plant cell into which it has been introduced, thereby providing the desired phenotypic trait to the plant comprised of the transformed cells. Expression of the protein in a plant cell is responsible for the altered characteristics of the cell, and consequently the characteristics of a plant comprised of the transformed cells.

One of skill in the art would know of a variety of vectors which can be used to express the DNA fragment of the present invention. Furthermore, methods of transforming plant cells, specifically cotton plant cells, most specifically cotton seed cells, are well known to one of skill in the art.

For example, one common method used to introduce foreign genes into plant cells is transformation with Agrobacterium, a relatively benign natural plant pathogen. Agrobacterium actively mediates transformation events—the integration of a gene providing a desired phenotypic trait—as part of the natural process it utilizes when it infects a plant cell. Methods for transferring foreign genes into plant cells and the subsequent expression of the inserted genes in plants regenerated from transformed cells are well known in the prior art. See for example, M. De Block et al., The EMBO Journal (1984) 3:1681; Horsch et al. Science (1985) 227:1229; and C. L. Kado (Crit. Rev. Plant. Sci. (1991) 10:1.

The technique known as microprojectile bombardment has been used to successfully introduce genes encoding new genetic traits into a number of crop plants, including cotton, maize, tobacco, sunflowers, soybeans and certain vegetables. See for example, U.S. Pat. No. 4,945,050, issued to Sanford; Sanford et al., Trends in Biotechnology (1988) 6:299; Sanford et al., Part. Sci. Technol. (1988) 5:27; J. J. Finer and M. D. McMullen, Plant Cell Reports (1990) 8:586-589; and Gordon-Kamm, The Plant Cell (1990) 2:603). Transformation by microprojectile bombardment is less species and genotype specific than transformation with Agrobacterium, but the frequencies of stable transformation events achieved following bombardment can be quite low, partly due to the absence of a natural mechanism for mediating the integration of a DNA molecule or gene responsible for a desired phenotypic trait into the genomic DNA of a plant. Particle gun transformation of cotton for example, has been reported to produce no more than one clonal transgenic plant per 100-500 meristems targeted for transformation. Only 0.1 to 1% of these transformants were capable of transmitting foreign DNA to progeny. See WO 92/15675. Cells treated by particle bombardment must be regenerated into whole plants, which requires labor intensive, sterile tissue culture procedures and is generally genotype dependent in most crop plants, particularly so in cotton. Similar low transformation frequencies have been reported for other plant species as well.

DNA to be inserted into the plant is generally in the form of a plasmid vector and is constructed using methodology known to those of skill in the art of plant molecular biology. Exemplary methods are described in Current Protocols In Molecular Biology, F. Ausubel et al. (eds.), Wiley Interscience (1990) and “Procedures for introducing foreign DNA into plants” in Methods in Plant Molecular Biology and Biotechnology, B. R. Glick, and J. E. Thompson, eds., CRC Press, Inc., Boca Raton, (1993).

The DNA to be expressed is flanked by suitable promoters known to function in plant cells, such as the 35S promoter from cauliflower mosaic virus (CaMV), described by Odell et al., Nature (1985) 313:810; or the nopaline or octopine synthetase promoters (NOS) from Agrobacterium, described by Vontling et al., Mol. Plant-Microbe Interactions (1991) 4:370; and M. de Block et al., The EMBO Journal (1984) 3:1681. Any promoter which functions in a plant can be used to express the gene encoding the desired trait, including inducible, tissue-specific, tissue-preferred or constitutive promoters. Other regulatory sequences such as transcription termination sequences, polyadenylation sequences, and intervening sequences, or introns, which provide enhanced levels of expression may also be included in the DNA construct or plasmid used for transformation. Depending upon the desired function of the gene, it may be desirable to include protein sequences which direct the secretion or intracellular compartmentalizations of the DNA to be expressed. Such sequences are well-known to those of skill in the art of plant molecular biology.

The plasmid may also contain a DNA sequence encoding a selectable marker gene or a screenable marker gene, which can be used to identify individual transformed plants. The marker may allow transformed plants to be identified by negative selection or by screening for a product encoded by a genetic marker. Suitable selectable markers include antibiotic and herbicide resistance genes such as the neomycin transferase gene (NPTII) described by Fraley et al., Proc. Natl. Acad. Sci. U.S.A. (1983) 80:4803 and by van den Elzen et al., Plant Mol. Biol., (1985) 5:299; the or the phosphinothricin acetyl transferase genes (pat and bar) described in U.S. Pat. Nos. 5,561,236 and 5,276,268. Markers which may be used to directly screen for transformed plants include the β-glucuronidase gene (GUS), the luciferase gene, the green fluorescence protein gene and the chloramphenicol acetyltransferase gene. R. G. Jefferson, Plant Molecular Biology Reporter (1987) 5:387; C. Koncz et al., Proc. Natl. Acad. Sci. (1987) 84:131; Teri et al., EMBO J. (1989) 8:343; and De Block et al., EMBO J. (1984) 3: 1681. Any gene encoding a selectable or screenable marker known to function in plant cells or plant tissues may be used in the method.

The abbreviations used are: CAD for (+)-d-cadinene synthase; COSY for Correlation Spectroscopy; GC for gas chromatography; MS for mass spectrum; NMR for nuclear magnetic resonance; DPA for days post anthesis; FDP for farnesyl diphosphate; RT for reverse transcription; and TMS for tetramethylsilane.

Cotton plants (Gossypium spp.) accumulate secondary sesquiterpenes in subepidermal glands of aerial tissues and in root epidermal cells. These defense compounds may also function as phytoalexins, with their formation induced by fungal and bacterial infection and by other stress factors (Bell et al (1986) In Natural Resistance of Plants to Pests (Green, M. A., and Hedin, P. A., eds.). Pp. 36-54, Amer. Chem. Soc., Washington, D.C.; Davila-Huerta et al (1995) Phytochemistry 39, 531-536; and Tan et al (2000) Planta 210, 644-651.). The majority of cotton secondary sesquiterpenoids, including gossypol, are derived from a common parent compound, (+)-ä-cadinene (4). The cotton (+)-ä-cadinene synthase (CAD1 or CDN1), a sesquiterpene cyclase, has been investigated at both enzymatic and molecular levels (Tan et al (2000) Planta 210, 644-651; Davis et al (1995) Phytochemistry 39, 553-567; Chen et al (1995) Arch Biochem Biophys. 324, 255-266; Chen et al (1996) J Nat Prod. 59, 944-951; Davis et al (1996) Phytochemistry 41, 1047-1055; Alchanati et al (1998) Phytochemistry 47, 961-967; Liu et al (1999) Mol. Plant Microbe Interact. 12, 1095-1104; and Meng et al (1999) J. Nat. Prod. 62, 248-252). Little is known about enzymes catalyzing subsequent biosynthetic steps. Desoxyhemigossypol-O-methyl transferase, which catalyzes one of the late steps, has been purified (Liu et al (1999) Plant Physiol. 121, 1017-1024). However, until now no enzymes that modify (+)-ä-cadinene itself have been reported.

Cytochrome P450 monooxygenases are enzymes that activate molecular oxygen and typically insert one oxygen atom, as a hydroxyl group, into lipophilic substrates (Halkier, B. A. (1996) Phytochemistry 43, 1-21). In plants, these enzymes participate in many biochemical pathways, including secondary metabolism, hormone biosynthesis and detoxification of xenobiotics (Chapple, C. (1998) Ann. Rev. Plant Physiol. Plant Mol. Biol. 49, 311-343). A number of P450s of the phenylpropanoid pathway have been cloned from plants (Chapple, C. (1998) Ann. Rev. Plant Physiol. Plant Mol. Biol. 49, 311-343; Jung et al (2000) Nature Biotechnol. 18, 208-212; Martens, S., and Forkmann, G. (1999) Plant J. 20, 611-618; Akashi, T. et al (1999) Plant Physiol. 121, 821-828; Humphreys et al (1999) Proc. Natl. Acad. Sci. U.S.A. 96, 10045-10050; and Steele et al (1999) Arch. Biochem. Biophys. 367, 146-50). In terpenoid pathways, P450 monooxygenases are involved in biosynthesis of various classes of compounds (Hefner et al (1996) Chem. Biol. 3, 479-89; and Hedden et al (1997) Annu. Rev. Plant Physiol. Plant Mol. Biol. 48, 431-460). Microsomes prepared from Mentha spp. were demonstrated to catalyze hydroxylation of the monoterpene (−)-4S-limonene (Karp et al (1990) Arch. Biochem. Biophys. 276, 219-226; Lupien et al (1995) Drug Metab. Drug Interact. 12, 245-260), and recently cDNAs encoding two regiospecific P450 limonene hydroxylases, (−)-4S-limonene-3-hydroxylase and (−)-4S-limonene-6-hydroxylase, were reported (Lupien et al (1999) Arch. Biochem. Biophys. 368, 181-192). For biosynthesis of taxol, a diterpenoid found in trees of Taxus spp., the first oxygenation step was found to be a P450-dependent reaction (Hefner et al (1996) Methods Enzymol. 272, 243-250; Hezari et al (1997) Planta Med. 63, 291-295). The P450s were also shown to be involved in resin biosynthesis of conifer trees (Funk et al (1994) Arch Biochem Biophys. 308, 258-266). Sesquiterpenes constitute the largest group of natural terpenoids (Bohlmann et al (1998) Proc. Natl. Acad. Sci. U.S.A. 95, 4126-4133), and P450s are also proposed to play a major role in sesquiterpene biosynthesis (Mihaliak et al (1993) Methods Plant Biochem. 9, 261-279). Although great progress has been made in investigation of plant sesquiterpene cyclases, which catalyze the first committed steps in secondary sesquiterpene biosynthesis (Bohlmann et al (1998) Proc. Natl. Acad. Sci. U.S.A. 95, 4126-4133; Chappell, J. (1995) Annu. Rev. Plant. Physiol. Plant Mol. Biol. 46, 521-547), up to now P450 enzymes catalyzing subsequent oxidative reactions of sesquiterpenes have not been characterized at the molecular level.

The cotton (+)-ä-cadinene synthase is encoded by a gene family. On the basis of sequence similarities, the family has been divided into two subfamilies, CAD1-A and CAD1-C. The diploid genome of G. arboreum contains about six members of CAD1-C, and a single copy of CAD1-A (Tanet al (2000) Planta 210, 644-651). Both CAD1-C and CAD1-A members are actively transcribed in developing seeds of glanded cotton cultivars, but neither are transcribed in seeds of a glandless cultivar, of which the seeds are gossypol free (Meng et al (1999) J. Nat. Prod. 62, 248-252). Therefore, there is reason to assume that genes coding for other enzymes in the gossypol pathway are also silent in developing seeds of glandless cultivars.

In connection with the present invention, a P450 cDNA was isolated from G. arboreum by using a combinatory strategy of PCR and differential hybridization. Microsomal proteins prepared from yeast cells expressing this P450 catalyzed hydroxylation of (+)-ä-cadinene in vitro. This cotton P450 has been placed in a new subfamily as CYP706B1, and it is the first member of the CYP706 family for which the function has been determined.

The present invention will be further understood with reference to the following examples.

### EXAMPLE 1

Materials

Plants of Gossypium arboreum L. cv. Qingyangxiaozhi, G. hirsutum L. cv. Zhong-12, and a glandless cultivar G. hirsutum cv. Hai-1 were grown in a greenhouse. Flowers, peels (pericarp of the cotton boll), and seeds were collected at various developmental intervals as previously described (Tan et al (2000) Planta 210, 644-651; and Meng et al (1999) J. Nat. Prod. 62, 248-252). Cell suspension cultures of G. arboreum cv. Qingyangxiaozhi were maintained in liquid MS medium (Murashige, J., and Skoog, F. (1962) Physiol. Plant. 115, 473-497), and transferred into fresh medium every seven days. Elicitors of the fungus Verticillium dahliae were prepared and applied to suspension cultured cells at a final concentration of 1 mg sucrose equivalent per mL culture, as previously described (Liu et al (1999) Mol. Plant Microbe Interact. 12, 1095-1104; Heinstein, P. (1985) J. Nat. Prod. 48, 907-915).

Cloning of cDNA—A Degenerate Primer

5ç-GCGGATCCGA(AG)TT(CT)(AC)G(AGCT)CC(AGCT)GA(AG)(AC)G (sense) was synthesized corresponding to a conserved peptide sequence of EEF(L/R)PERF (Frank et al (1996) Plant Physiol. 110, 1035-1046), about 20 amino acids upstream of the heme-binding domain of plant P450 monooxygenases. It was used together with a vector-specific T7 primer (reverse; Stratagene, La Jolla, Calif.), in PCR amplification of P450 fragments from a 1-UniZap cDNA library constructed from elicitor-treated G. arboreum cells (Chen et al (1995) Arch Biochem Biophys. 324, 255-266). The PCR program was: 94° C. for 30 s, 55° C. for 30 s, 72° C. for 30 s; 30 cycles. The PCR products of were inserted into pGEM-T vectors (Promega, Madison, Wis.). After amplification in E. coli, plasmid DNA from individual clones was spotted onto nitrocellulose membranes, which were then baked at 80° C. for 2 hrs for subsequent hybridization screening.

Probes were generated from total RNAs isolated from developing seeds of the glanded and glandless G. hirsutum cultivars, respectively, and the first strand cDNA was synthesized as previously described (Meng et al (1999) J. Nat. Prod. 62, 248-252). The cDNAs were ³²P-labeled using a random DNA labeling kit (Takara, Dalian, China), and used for dot-hybridizations. Clones LP132 and LP64 showing preferential hybridization with probes of glanded seeds were selected and sequenced by the dideoxynucleotide chain termination method. Specific primers LP132F [5ç-TGACTGATCATGAGAAGCT (sense)] and LP132R [5ç-GTGCTGGAGATIRGATGGT (reverse)] based on the sequence of LP132 were then used for screening the G. arboreum cDNA library by using a PCR 96-well plate method (Liu et al (1999) Mol. Plant Microbe Interact. 12, 1095-1104). A cDNA clone, CYP706B1, was then isolated and sequenced (See SEQ. ID. NO. 1; GenBank/EBI Data Bank Accession No. AF332974).

DNA and RNA Analysis

Genomic DNA of G. arboreum was isolated from foliar tissues as described (Tan et al (2000) Planta 210, 644-651). After complete digestion (4 hrs to overnight) with restriction enzymes of EcoR I, Xba I and EcoR V, about 20 mg of DNA per lane were separated by electrophoresis and transferred onto a nitrocellulose membrane. For probe preparation, the CYP706B1 was digested with Xba I and EcoR V, and the 947 bp fragment released was ³²P labeled. Hybridization and washing were performed following a standard protocol (Sambrook, J., Fritsch, E. F., and Maniatis, T. (1989) Molecular Cloning: A Laboratory Manual. (2nd ed.). Cold Spring Harbor Laboratory Press, NY.), and the membrane was finally exposed to X-ray film for 1^{˜}2 days.

Pericarp (approx. 3 mm thick) was peeled from bolls with a blade. Total RNAs were isolated from tissues or from suspension cultured cells by a cold phenol method, and the transcripts were analyzed by RT-PCR with primers LP132F and LP132R for CYP706B1 (position 1433^{˜}1689), 97400 [5ç-CACATCC(AC)TTCGATTCCGAC (sense)] and 97T580 [5ç-AGGCTTAAATGGTGGGTGGT (reverse)] for CAD1-C (position 398^{˜}610), and H3F [5ç-GAAGCCTCATCGATACCGTC (sense)] and H3R [5ç-CTACCACTACCATCATGTC (reverse)] for the histone gene his3 (positions 95^{˜}526). For Northern analysis, 10 mg of RNA per lane were separated by electrophoresis, blotted onto a nitrocellulose membrane, and the blots were hybridized with ³²P labeled DNA probes of either CYP706B1 (see above) or CAD1-C1 (Liu et al (1999) Mol. Plant Microbe Interact. 12, 1095-1104). After hybridization and washing, the blots were exposed to X-ray film for 2 days.

Expression in Yeast Cells and Enzyme Assay

The yeast Saccharomyces cerevisiae strain W(R), which overexpresses the yeast cytochrome P450 reductase when grow on galactose, and the expression vector pYeDP60 were provided by D. Pompon (Pompon et al (1996) Methods Enzymol. 272, 51-64). The cDNA of CYP706B1 was modified by PCR with a 5′-terminal primer 5ç-GGGTACCATGTTGCAAATAGCTTTCAG (sense), in which a Kpn I site was introduced, and a 3′-terminal primer 5ç-GGGAGCTCTTACTTCATATAGTGCTGGA (reverse), in which a Sac I site was introduced. PCR was conducted on plasmid DNA by using Pyrobest™ DNA polymerase (TaKaRa). After digestion with the restriction enzymes, the fragment was inserted into pYeDP60. Plasmid DNA was introduced into yeast cells by a LiAc method, transformed yeast cells were then selected, cultured, and induced, and microsomes were prepared following a high density procedure (Pompon et al (1996) Methods Enzymol. 272, 51-64).

The (+)-d-cadinene hydroxylase activity assay was based on a published protocol for monoterpene P450 hydroxylases (Mihaliak et al. (1993) Methods Plant Biochem. 9, 261-279). The radioactive assay was conducted in 100 mL of potassium phosphate buffer (50 mM, pH 7.4) containing 1 mM EDTA (pH 8.0), 0.4 M sucrose, 2 mM DTT, 0.1% BSA, 1 mM NADPH, 5 mM FAD, 5 mM FMN, 4 mM glucose-6-phosphate, 1 unit of glucose-6-phosphate dehydrogenase, 40 mM ³H-(+)-d-cadinene (32 mCi/mmol) and yeast microsomes (100^{˜}200 mg protein). The reaction was started by adding the microsomes, incubated at 30° C. for 1 hr, and stopped by chilling on ice. The reaction mixture was extracted three times with 500 mL hexane:ethyl acetate (1:1), the extract was filtered through a 0.2 g, 100^{˜}200 mesh silica gel column in a pasteur pipet, eluted with 1.5 mL hexane:ethyl acetate (1:1), and concentrated to ca 100 mL with an argon stream. The entire sample was subjected to reversed phase HPLC on C₁₈ silica (250×4.6 mm, 5 mm particle diameter) with a gradient of 40:60 to 100:0 acetonitrile/water (v/v) over 60 min followed by 10 min of 100% acetonitrile, flow rate 1 mL/min. Tritium was detected by an on-line liquid scintillation counter (b-RAM, IN/US Systems, Inc., Tampa, Fla.). The tritium-labeled product eluted at 33^{˜}34 min, followed by the substrate, (+)-d-cadinene, at 59^{˜}60 min.

[³H](+)-d-cadinene was prepared from commercial (E,E)-[1-³H]farnesyl diphosphate (FDP, NEN, Boston, Mass.), with the CAD1-C1 fusion protein expressed in E. coli and prepared as described (Chen et al (1995) Arch Biochem Biophys. 324, 255-266), except that the sonication buffer was 100 mM Tris/HCl, pH 7.4, 1 mM EDTA, 0.1% Tween 20. The reaction mixture consisted of 1 mL of the E. coli extract (ca 7 mg protein), 8 mL of 30 mM HEPES, pH 7.0, 10% glycerol, 1 mM MgCl₂, and 1 mL of 1 mM [³H]FDP (diluted to a specific activity of 32 mCi/mmol with non-radioactive (E,E)-FDP in 25 mM (NH₄)₂CO₃ (pH 7.0), 40% glycerol (35), degassed with argon. The reaction mixture was incubated at 30° C. with gentle shaking for 60 min. Products were extracted at 4° C. with hexane (4×10 mL), applied to a silica gel column (2 g, 100^{˜}200 mesh) to remove any [1-⁻³H]farnesol, and eluted with 20 mL hexane. The eluate was added to 5 mL 2-propanol and evaporated in vacuo to ca 3 mL. Purity of the resulting [³H](+)-d-cadinene was checked by HPLC as described above.

Protein concentration was determined with the Bio-Rad Protein Assay (Bio-Rad, Hercules, Calif.). Sesquiterpene aldehydes were extracted and quantitated by the phloroglucinol method (36).

Isolation of the Reaction Product

Samples for GC-MS and NMR analysis were prepared from non-radioactive (+)-d-cadinene, which had been prepared by acid-catalyzed rearrangement of commercial (−)-a-cubebene (Davis et al (1995) Phytochemistry 39, 553-567). To avoid problems with scaling up the reaction, one hundred 1.0-mL reaction mixtures (same components and concentrations as for the (+)-d-cadinene hydroxylase activity assay above) were prepared with 200 mM non-radioactive (+)-d-cadinene as substrate and incubated at 30° C. for 30 minutes. The reaction was quenched by adding 0.2 mL diethyl ether to each tube. The mixtures were combined, the ether was removed, and products were extracted from the aqueous phase twice more with 20 mL diethyl ether. The combined ether extract was dried by passage through a silica gel (1 g, 10^{˜}40 mm particle size, 9 mm×30 mm) column, and evaporated to yield an 11.4 mg residue. The residue was dissolved in 100 mL of diethyl ether and chromatographed on a silica gel (3 g, 10^{˜}40 mm particle size, 9 mm×90 mm) column with hexane-ethyl ether (4:1, v/v) as eluant, collecting 2-mL fractions. Fractions 7 to 15 were combined and washed with 3 M sodium carbonate buffer, pH 12.

Identification of the Reaction Product

Samples were analyzed for 8-hydroxy-(+)-d-cadinene using GC-MS on a HP 5890 series II GC equipped with a HP-ZB5 column (30 m×0.25 mm). The helium inlet pressure was controlled by Electronic Pressure Control to achieve a constant column flow of 1.0 ml/min during the following oven program: initial temp. 45° C. for 5 min, ramp of 5° C./min to 295° C. and 10 min at 295° C. Ionization potential was 70 eV. ¹H NMR and ¹H-¹H COSY spectra were recorded on a Varian Unity Inova 600 spectrometer using TMS as internal standard.

cDNA Cloning and Analysis

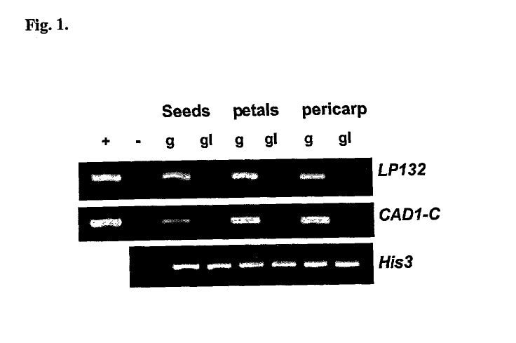

The 3ç-terminal cDNA fragments of P450 monooxygenases were amplified by PCR from a G. arboreum library that was constructed from mRNAs of fungal elicitor-treated suspension cells (Chen et al (1995) Arch Biochem Biophys. 324, 255-266). This resulted in a mixture of DNA fragments with different lengths ranging from 300^{˜}600 bp, possibly due to a large number of P450 genes expressed in cotton cells after elicitation. About 100 individual clones of the PCR products were then used in differential dot-blot hybridization, with cDNA probes prepared from developing seeds of a glanded cultivar G. hirsutum cv. Zhong-12, and of a glandless cultivar G. hirsutum cv. Hai-1, respectively. Two clones with an approximately 500 base-pair insert, LP132 and LP64, showed clear hybridization signals with glanded probes only (data not shown), and their nucleotide sequences were then determined. A search of the GenBank database with the NCBI blastx program revealed high sequence similarities between LP132 and plant P450 monooxygenases (30^{˜}45% identity at the amino acid sequence level). Subsequent RT-PCR with primers specific to LP132 indicated that, while a significant level of its transcript was present in developing seeds, petals and pericarp of the glanded cultivar, this transcript was indeed undetectable in those tissues of the glandless cultivar. As shown in FIG. 1, and also as reported previously (Tan et al (2000) Planta 210, 644-651; Meng et al (1999) J. Nat. Prod. 62, 248-252), this expression pattern was identical to that of (+)-d-cadinene synthase, a sesquiterpene cyclase that catalyzes the first committed step in biosynthesis of cotton sesquiterpene phytoalexins.

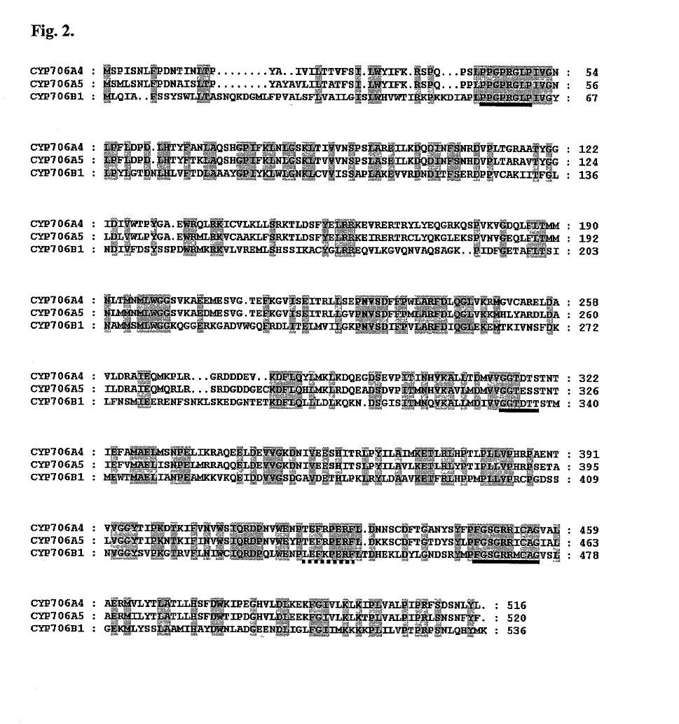

A cDNA clone corresponding to LP132 was isolated from the G. arboreum library. It contained a reading frame coding for a protein of 536 amino acid residues, with a calculated molecular mass of 60.12 kDa. Its alignment with known P450 cytochromes suggests that it contains the full-length coding sequence. An NCBI blast search of the GenBank database with its deduced protein sequence revealed highest sequence identity (up to 47%) with six putative P450 proteins from Arabidopsis thaliana (CYP706A1 through CYP706A6; AL080318, genomic sequences of chromosome 4). Its alignment with its two closest homologues, CYP706A4 and CYP706A5, is shown in FIG. 2. Among proteins of known functions, it has the highest amino acid sequence identity (34.2%) with a flavonoid 3′-hydroxylase from Petunia x hybrida, a member of CYP75B2 (Brugliera et al (1999) Plant J. 19, 441-451), followed by 32% with a flavonoid 3′,5′-hydroxylase from Solanum melongena, a member of CYP75A2 (Toguri et al (1993) Plant Mol. Biol. 23, 933-946). This cotton P450 has been placed in a new subfamily as CYP706B1 (accessed via dnelson@utmenl.utmen.edu).

Sequence analysis revealed several structural motifs characteristic of eukaryotic P450s (FIG. 2). The highly conserved heme-binding motif FxxGxRxCxG (Chapple, C. (1998) Ann. Rev. Plant Physiol. Plant Mol. Biol. 49, 311-343) was found in CYP706B1 as FGSGRRMCAG, 73 amino acid residues from the C-terminus. In most plant P450s, there is a proline residue immediately after the invariant heme-binding cysteine (Schalk et al (1999) Biochemistry 38, 6093-61103); however, in CYP706B1, this proline is replaced by alanine. The proline-rich region immediately after the N-terminal signal anchor sequence (Nelson, D. R., and Strobel, H. W. (1988) J. Biol. Chem. 263, 6038-6050), with a consensus of (P/I)PGPx(G/P)xP (Schalk et al (1999) Biochemistry 38, 6093-61103), was completely conserved in this cotton P450 as PPGPRGLP. In addition, the threonine-containing pocket for binding an oxygen molecule, with a consensus of (A/G)Gx(D/E)T(T/S) (Durst, F., and Nelson, D. R. (1995) Drug Metab. Drug Interact. 12, 189-206), was also found (as GGTDTT).

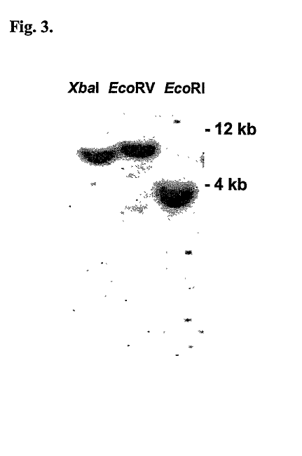

Hybridization of the genomic DNA of G. arboreum with a CYP706B1 probe revealed a single band in EcoR I, EcoR V and Xba I digested DNA samples, respectively (FIG. 3). This hybridization pattern indicated a single copy gene encoding CYP706B1 in the genome of G. arboreum, a diploid cotton species.

Sesquiterpene Hydroxylase Activities

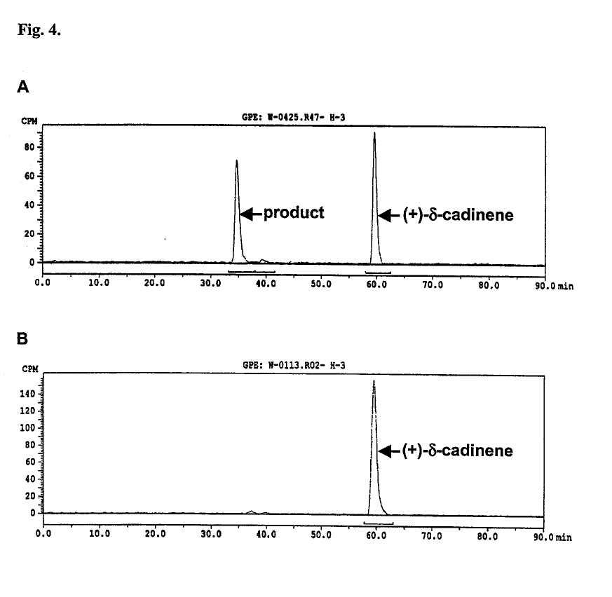

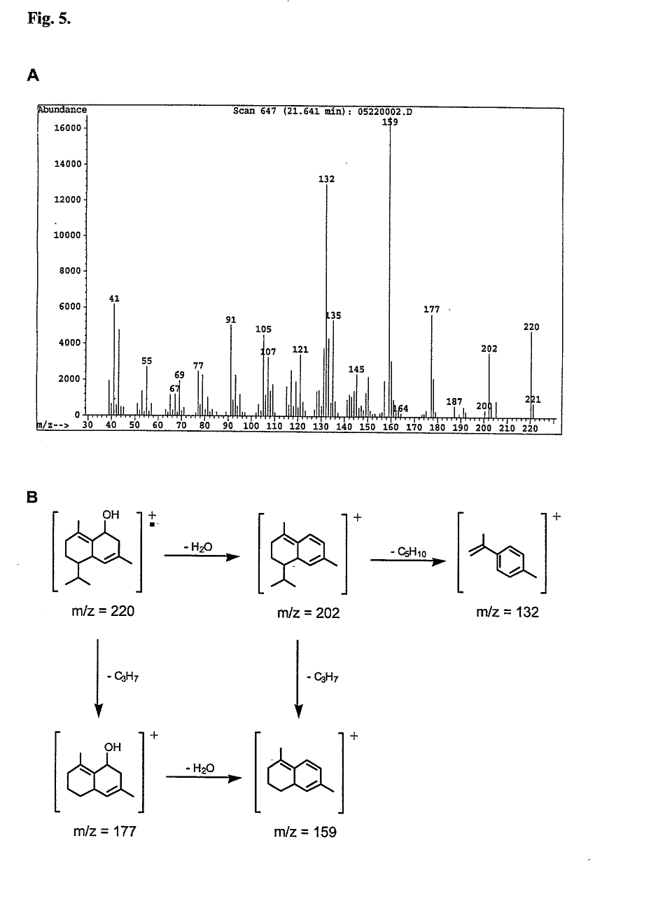

Microsomal proteins prepared from yeast cells expressing CYP706B1 showed clear hydroxylase activities in vitro with tritium-labeled (+)-d-cadinene as a substrate. HPLC revealed a single product peak with more polarity than the (+)-d-cadinene (FIG. 4A). When the microsomes from yeast cells harboring a different P450-like clone, LP64 (FIG. 4B), or from yeast cells harboring an empty pYeDP60 vector (data not shown), were used as the catalyst, no product was detected by radio-HPLC. In a 15 min assay, the yeast microsomes containing CYP706B1 showed a specific activity of 42.6±2.8 nmol product/mg protein h. Highest activity was achieved when the reaction was supported by 1 mM NADPH; when replaced by NADH, the activity decreased by about 65%. Treating the reaction mixture with a slow stream of CO for about 2 min inhibited the hydroxylase activity by 70%.

Product Identification by GS/MS and NMR

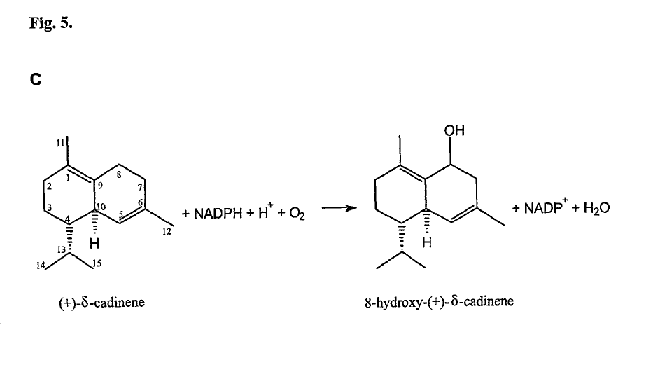

GC analysis also revealed a single peak of product (data not shown). The mass spectrum of the product showed a molecular mass of 220 (FIG. 5A), consistent with that of an 8-hydroxy-d-cadinene (molecular mass of (+)-d-cadinene is 204.). The MS fragmentation pattern exhibited the loss of water, loss of the isopropyl group (C₃H₇), and reverse Diels-Alder cleavage (—C₅H₁₀) that are typical of sesquiterpenes with this carbon skeleton (FIG. 5B) (Davis, G. D., and Essenberg, M. (1995) Phytochemistry 39, 553-567). The ¹H NMR spectrum was very similar to that of d-cadinene (Table 1).

TABLE I
¹H NMR chemical shifts of d-cadinene and 8-hydroxy-d-cadinene

| H | d-cadinene¹ | 8-hydroxy-d-cadinene² |
|---|---|---|
| H₂-2 | 1.95 (m) | |
| H-2a | | 2.02 (br.d, 12.1 Hz) |
| H-2b | | 1.96 (br.t, 7.2 Hz) |
| H-3a | 1.61 (m) | 1.63 |
| H-3b | 1.16 (m) | 1.16 |
| H-4 | 1.05 (m) | 1.12 (m) |
| H-5 | 5.45 (br.s) | 5.60 (br.s) |
| H₂-7 | 2.00 (m) | |
| H-7a | | 2.40 (br.d, 12.5 Hz) |
| H-7b | | 2.21 (br.d, 12.5 Hz) |
| H-8a | 2.72 (m) | 3.45 |
| H-8b | 1.90 (m) | |
| H-10 | 2.52 (br.d, 9.0 Hz) | 2.25 (d, 12.2 Hz) |
| H-13 | 2.06 (m) | 1.91 |
| 11-Me | 1.66 (s) | 1.67 (s) |
| 12-Me | 1.68 (s) | 1.69 (s) |
| 14-Me | 0.96 (d, 6.9 Hz) | 1.10 (d, 6.8 Hz) |
| 15-Me | 0.78 (d, 6.9 Hz) | 1.00 (d, 6.8 Hz) |

¹ 400 MHz, CDCl₃ (Davis et al, 1996). ² 600 MHz, d₆-benzene. Signal multiplicities are indicated as follows: m: multiplet; br.s: broad singlet; s: singlet; d: doublet; br.t: broad triplet.

The differences in chemical shifts and multiplicities indicated that the product is hydroxylated at C-8. A two-dimensional ¹H-¹H COSY analysis revealed all the expected connectivities between hydrogen atoms on the same or adjacent carbon atoms except that for H-13 to H-4. To the best of our knowledge, this is the first report of 8-hydroxy-(+)-d-cadinene. The reaction catalyzed by CYP706B1 of Gossypium arboreum is given in FIG. 5C. Some preparations of the product appear to be conjugated through the 8-hydroxyl group to a moiety that has not yet been identified.

Expression Pattern

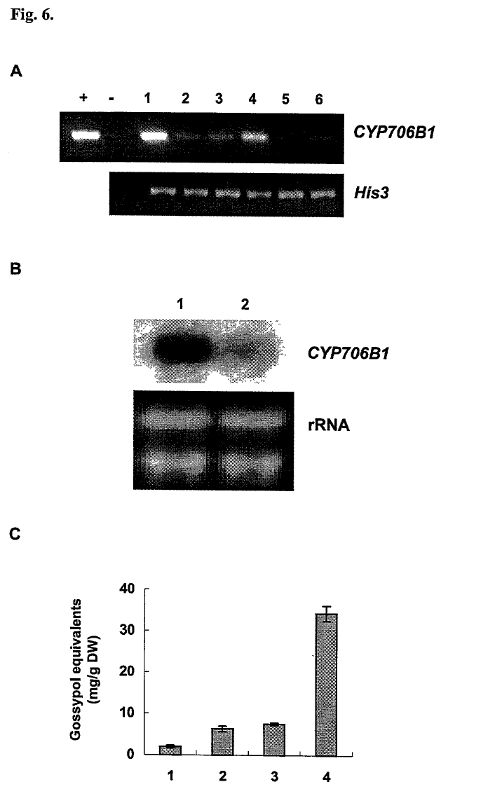

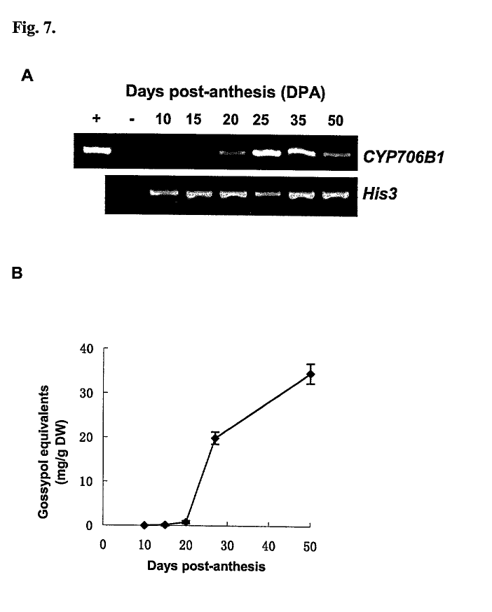

In cotton roots, gossypol and related sesquiterpene aldehydes are stored in epidermal tissues, rather than in subepidermal glands, as found in aerial tissues. RT-PCR indicated that CYP706B1 was expressed in roots of both the glanded G. arboreum and glandless cultivar of G. hirsutum cv. Hai-1 (FIG. 6A), although, according to Northern analysis, roots of the glanded cultivar had a higher steady-state mRNA level than roots of the glandless cultivar (FIG. 6B). Similarly, roots of G. arboreum had a higher level of sesquiterpene aldehydes than roots of G. hirsutum cv. Hai-1 (FIG. 6C). Transcripts of CYP706B1 were also detected in cotyledons and hypocotyls of G. arboreum seedlings by RT-PCR, but not in cotyledons and hypocotyls of the glandless G. hirsutum cv. Hai-1 (FIG. 6A). Accordingly, sesquiterpene aldehydes were detected in glanded cotyledons and hypocotyls (FIG. 6C), but not in glandless cotyledons and hypocotyls (data not shown). In developing seeds of G. arboreum, CYP706B1 transcripts were detected at 20 DPA and afterwards (FIG. 7A) followed by sesquiterpene aldehyde accumulation (FIG. 7B), a pattern similar to CAD1-C expression and sesquiterpene accumulation in developing seeds of another glanded cultivar, G. hirsutum cv. Sumian-6 (Meng et al (1999) J. Nat. Prod. 62, 248-252).

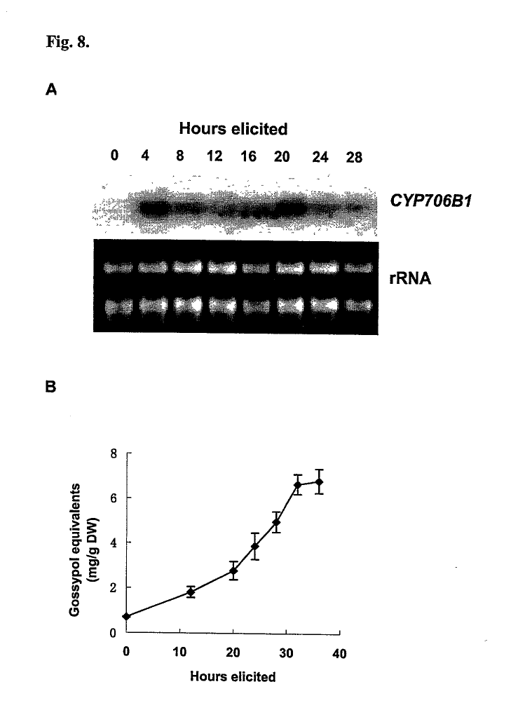

When suspension cultured cells of G. arboreum were treated with elicitors of Verticillium dahliae, a phytopathogenic fungus responsible for a vascular wilt disease of cotton, transcription of CYP706B1 was significantly induced (FIG. 8A), followed by increased production of sesquiterpene phytoalexins (FIG. 8B). After a quick induction of the CYP706B1 transcription within 4 hours of elicitation, the mRNA steady-state level peaked again around 20 hours post-elicitation (FIG. 8A). The elicitation experiment was then repeated and the double peaks of the transcription level were again detected by Northern hybridization (data not shown).

According to hydroxylation positions, there are two groups of cadinane-type sesquiterpenoids in cotton. The 7-hydroxylated cadinanes, such as 2,7-dihydroxycadalene and lancinilene C, are induced to accumulate in foliar tissues after bacterial infection (Davila-Huerta et al (1995) Phytochemistry 39, 531-536; Essenberg et al (1990) Phytochemistry 29, 3107-3113;

Pierce et al (1996) Physiol. Mol. Plant Pathol. 48, 305-324). The 8-hydroxylated cadinanes, such as gossypol and related sesquiterpene aldehydes, are the largest group of cotton secondary sesquiterpenoids and are distributed in roots, seeds and glanded green tissues; in addition, their formation may also be elicited by fungal or bacterial infection (Bell et al (1986) In Natural Resistance of Plants to Pests (Green, M. A., and Hedin, P. A., eds.). Pp. 36-54, Amer. Chem. Soc., Washington, D.C.; Tan et al (2000) Planta 210, 644-651; and Essenberg et al (1990) Phytochemistry 29, 3107-3113). Hydroxylation of (+)-d-cadinene at C-8 is theorized to be the second step in the biosynthetic pathway leading to gossypol; therefore cloning of an enzyme catalyzing this hydroxylation of cadinene is useful in elucidation and investigation of gossypol biosynthesis and in the suppression of gossypol formation through genetic manipulation.

In various tissues of seedlings and mature plants, the expression pattern of CYP706B1 reported herein is similar to that of sesquiterpene cyclase CAD1-C1 (Tan et al (2000) Planta 210, 644-651; and Meng et al (1999) J. Nat. Prod. 62, 248-252). In suspension cultured cells, expression of CYP706B1 is inducible by fungal elicitors (FIG. 8), as are FDP synthase and CAD1 (Chen et al (1995) Arch Biochem Biophys. 324, 255-266; Chen et al (1996) J Nat Prod. 59, 944-951; and Liu et al (1999) Mol. Plant Microbe Interact. 12, 1095-1104), the two enzymes immediately upstream of this P450 monooxygenase. These enzymes may be concordantly regulated, directing isoprenoids into sesquiterpene aldehydes, including gossypol. Coordinate regulation of enzymes involved in phenylpropanoid phytoalexin synthesis has also been reported (Logemann et al (2000) Proc. Natl. Acad. Sci. U.S.A. 97, 1903-1907).

Many plant P450s have been cloned by PCR amplification, however, functions of many genes obtained by this approach remain unknown (Chapple, C. (1998) Ann. Rev. Plant Physiol. Plant Mol. Biol. 49, 311-343). When used in combination with analysis of mutants, the method has been proven successful and efficient in isolation and identification of targeted P450s. Examples include flavonoid 3′,5′-hydroxylase from Petunia hybrida (Holton et al (1993) Nature 366, 276-2799), flavone synthase II from Gerbera hybrids (Martens, S., and Forkmann, G. (1999) Plant J. 20, 611-618), and the hydroxylase reported therein. Since genes encoding the first two enzymes in the gossypol pathway were found not to be expressed in developing seeds and other aerial tissues of healthy plants of G. hirsutum cv. Hai-1, this glandless mutant will be valuable for cloning other enzymes involved in gossypol biosynthesis. It seems that in cotton plants, common factor(s) control development of glands and biosynthesis of secondary sesquiterpenes. However, in roots, which accumulate sesquiterpenes in epidermal cells rather than in subepidermal glands, expression of CYP706B1 and biosynthesis of sesquiterpene aldehydes were detected in both the glanded and glandless cultivars. This suggests that mechanisms regulating secondary sesquiterpene biosynthesis in roots and in aerial tissues are at least partly different.

The cotton (+)-d-cadinene 8-hydroxylase is the first member of the CYP706 family whose function has been discovered. Among plant P450s, it is not closely related to monoterpene hydroxylases from Mentha (Lupien et al (1999) Arch. Biochem. Biophys. 368, 181-192), with only about 30% sequence identities. This is a contrast with plant terpene synthases of different classes, which share a common origin (Bohlmann et al (1998) Proc. Natl. Acad. Sci. U.S. A. 95, 4126-4133). Although phylogenetic analyses of P450 sequences throughout biological kingdoms indicate a common origin for cytochromes P450 (Yoshida et al (2000) Biochem. Biophys. Res. Commun. 273, 799-804), sequences may have diverged in different angiosperm families, followed by convergent evolution of substrate specificities, resulting in the monoterpene and sesquiterpene hydroxylases we now observe that have sequences of low similarity despite similar substrate specificities and identical function. Exploring functions of other CYP706 members, especially those of CYP706A of the model plant Arabidopsis thaliana, may shed light upon the relative evolution of amino acid sequence and of function within this subgroup.

Cottonseeds contain on the average 30% oil and over 30% protein, and are potentially useful as a foodstuff additive. However, their nutritional value is limited because of a high content of sesquiterpene aldehydes, mainly gossypol, which are toxic to monogastric animals. CYP706B1 appears to catalyze the second step in gossypol biosynthesis, directing (+)-d-cadinene into toxic sesquiterpene aldehydes. In addition, it is encoded by a single copy gene in the diploid G. arboreum. In comparison with (+)-d-cadinene synthase, which is encoded by a complex gene family, this enzyme provides a better target for suppression of gossypol formation in cottonseeds through genetic engineering.

While the invention has been described with a certain degree of particularity, it is understood that the invention is not limited to the embodiment(s) set for herein for purposes of exemplification.

We claim:

1. A DNA fragment comprising the sequence of SEQ ID NO:1.

2. A polypeptide comprising the amino acid sequence designated as CYP706B1 in FIG. 2.

3. A vector for transforming cotton comprising a DNA fragment which comprises the sequence of SEQ ID NO:1.
